(19) United States
(12) Patent Application Publication
(10) Pub. No.: US 2016/0015415 A1
(43) Pub. Date: Jan. 21, 2016

(54) ARTICULATING TISSUE DISSECTOR

(71) Applicant: Spineology Inc., St. Paul, MN (US)

(72) Inventor: Kyle Wolff, Cottage Grove, MN (US)

(21) Appl. No.: 14/802,948

(22) Filed: Jul. 17, 2015

Related U.S. Application Data
(60) Provisional application No. 62/025,837, filed on Jul. 17, 2014.

(51) Int. Cl. A61B 17/32

(52) U.S. Cl. CPC A61B 17/320016

(57) ABSTRACT

A tissue dissector can include a tool body having a proximal end and a distal end. The tool body can include a longitudinal path therein between the proximal end and the distal end. The path can include an arcuate portion disposed adjacent the distal end and a horizontal portion extending proximally from the arcuate portion. A cutting blade can be pivotably attached to the body with via a first pin. A central shaft can be coupled to the first pin. A handle assembly can be coupled to the central shaft. A second pin can be fixed to the cutting blade, engage the path of the tool body and be located offset from the first pin to pivot the blade about the first pin as the blade moves in a proximal direction from a farthest distal extent of the path in the tool body.

### PRIORITY

This application claims the benefit of U.S. Provisional Application Ser. No. 62/025,837, filed on Jul. 17, 2014, which is hereby incorporated herein by reference in its entirety.

### FIELD

The present invention generally relates to tissue dissectors. More particularly, the present invention relates to an articulating tissue dissector useful for creating a controlled cut.

### BACKGROUND

A variety of tools are available for surgeons for dissecting tissue. However there is a continuing need to provide improved tools for the same, especially for creating a controlled cut delivering consistent results with every use.

### SUMMARY

In one embodiment, the tissue dissector cuts a consistent amount of tissue each time to ensure proper rod insertion when using posterior fixation in a spinal surgery.

Disclosed is a tissue dissector comprising a tool body having a proximal end and a distal end, and defining a J-shaped path between the proximal and distal ends. A blade is movably attached to the distal end of the tool body. A handle can be coupled to the proximal end of the tool body, which actuates the blade such that the blade rotates up to 90 degrees out from a longitudinal axis of the tool body along the J-shaped path to create a J-shaped pathway in a patient's tissues.

Also disclosed is a tissue dissector comprising a tool body having a proximal end and a distal end. The tool body can include a longitudinal path therein between the proximal end and the distal end. The path includes an arcuate portion disposed adjacent the distal end and a horizontal portion extending proximally from the arcuate portion. A cutting blade is pivotably attached to the body with via a first pin. A central shaft is coupled to the first pin. A handle assembly is coupled to the central shaft. A second pin is fixed to the cutting blade, is engages the path of the tool body and is located offset from the first pin to pivot the blade about the first pin as the blade moves in a proximal direction from a farthest distal extent of the path in the tool body.

Further disclosed is a method of dissecting tissues of a patient. The method can include pivoting a cutting blade about a first pivot pin from a horizontal alignment to an angular offset alignment from the vertical alignment by moving a second pin coupled to the cutting blade through an arcuate portion of a path in a tool body, and moving the cutting blade in the horizontal plane in a proximal direction from the arcuate portion of the path while maintaining the cutting blade in the angular offset alignment by moving the second pin through a horizontal portion of the path in the tool body.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a side view of an embodiment of the present invention.

FIG. 2 depicts another side view of an embodiment of the present invention.

FIG. 3 depicts a further view of an embodiment of the present invention.

FIG. 4 depicts a side view of an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

The present invention is directed to a tissue dissector which may be used with a pedicle screw system, such as is disclosed in co-pending U.S. patent application Ser. No. 14/516,547, filed Oct. 16, 2014 and published as U.S. Pub. No. 2015/0105832 Al on Apr. 16, 2015. Said application is hereby incorporated herein by reference in its entirety. In an example embodiment, the dissector of the present invention may be placed down an extension guide that is connected to a tower. The dissector may be used to clear a pathway for the insertion of a rod and rod holder.

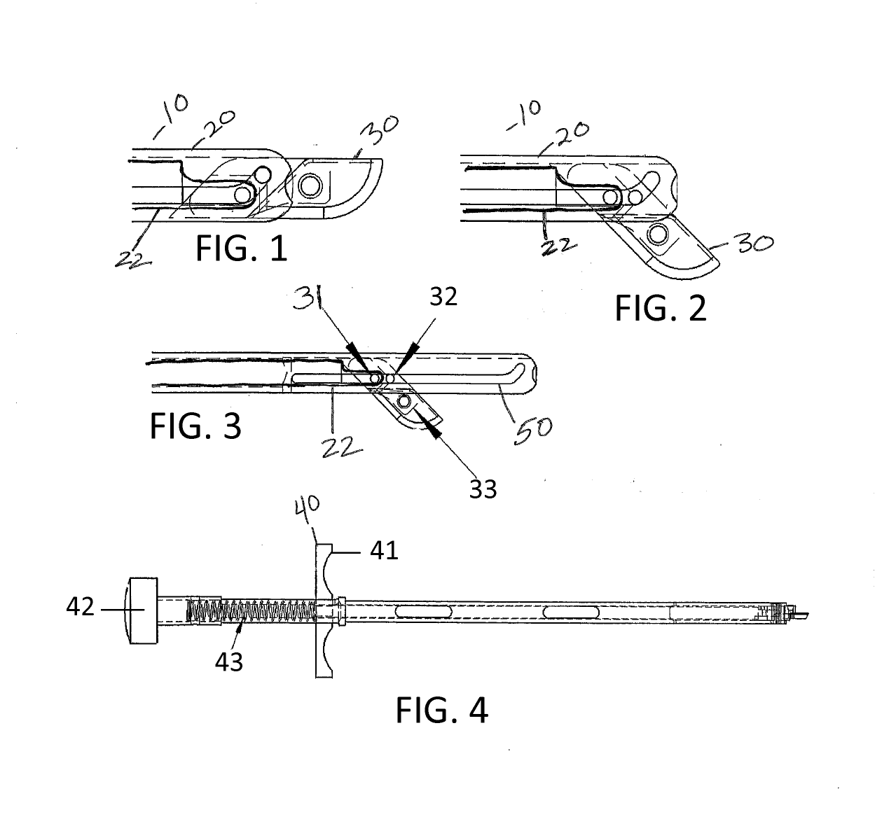

Referring to FIGS. 1-4, the tissue dissector 10 includes a tool body 20 having a proximal end and a distal end. A tool slot or path 50 is defined in the tool body. The path 50 includes a horizontal portion and a distal arcuate portion. The horizontal portion is aligned along the longitudinal extension of the tool body 20. The arcuate portion is defined adjacent the distal end of the tool body 20.

In an embodiment of the present invention, a blade 30 is be movably connected to the tool body 20 via a central shaft 22 and a plurality of pins.

A first pin 31 is a drive pin and is connected to the central shaft 22 so that the blade can be moved via the handle 40 assembly.

A second pin 32 acts as a follower to cause the blade 30 to pivot downwards at an oblique angle to the tool body 20 as the second pin 20 traverses the arcuate portion of the path 50.

A third pin is disposed laterally though the blade 30 so and acts as a cam surface riding along the bottom of the tool body 20 so that the blade's angle can be maintained even though a force component normal to the tool body's longitudinal surface is being applied during a cutting procedure.

The blade articulation is defined by the path 50 and placement of the first and second pins. For example, the path 50 can be generally J-shaped path as depicted in FIG. 3. Such J-shaped pathway 50 may serve as a template for the dissector 10 to cut and create a path for a rod insertion. Moving the blade through a precise and defined path allows the surgeon to precisely and repeatably remove a consistent amount of tissue from the patient in preparation for rod placement.

The proximal end of the dissector 10 can include a handle assembly 40 configured to actuate blade 30 by pulling the finger grip 41 in the proximal direction towards a palm stop 42. The central shaft 22 is coupled to the finger grip 41. Thus, the user can operate the tool with on e hand by using their fingers on one hand to pull the finger grip 41 towards the palm stop 42, which causes the blade to move as explained herein.

A resilient member 43, such as a spring, can be provided that is compressed as the blade is articulated, which will then return the blade to its initial state from any partially articulated state or the fully articulated state.

In use, the tool 10 can be inserted with the blade 30 in a horizontal position (as shown in FIG. 1) in-line with the longitudinal axis of tool body 20. Then proximal movement of the finger grip 41 of the handle causes the blade to pivot downwards (as shown in FIG. 2.) due to the first 31 and second 32 pins following the arcuate portion of the path 50. Further proximal movement of the finger grip 41 then moves the blade in a horizontal plane in a proximal direction. Releasing the finger grip 41 causes the spring 43 to expand and move the blade in the reverse direction until it stops at the zero degree or neutral position as shown in FIG. 1, where the blade 30 is horizontally aligned with the body.

In alternative embodiments, blade 30 may be rotated up to 90 degrees from the longitudinal axis of tool body 20 (e.g. from horizontal to vertical orientations).

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A tissue dissector, comprising:
a tool body having a proximal end and a distal end, and defining a J-shaped path between the proximal and distal ends;
a blade movably attached to the distal end of the tool body; and
a handle coupled to the proximal end of the tool body, which actuates the blade such that the blade rotates up to 90 degrees out from a longitudinal axis of the tool body along the J-shaped path to create a J-shaped pathway in a patient's tissues.

2. The tissue dissector of claim 1, further comprising means for rotating the blade.

3. A tissue dissector, comprising:
a tool body having a proximal end and a distal end, the tool body including a longitudinal path therein between the proximal end and the distal end, the path including an arcuate portion disposed adjacent the distal end and a horizontal portion extending proximally from the arcuate portion;
a cutting blade pivotably attached to the body with via a first pin;
a central shaft coupled to the first pin;
a handle assembly coupled to the central shaft;
a second pin fixed to the cutting blade, engaging the path of the tool body and located offset from the first pin to pivot the blade about the first pin as the blade moves in a proximal direction from a farthest distal extent of the path in the tool body.

4. The tissue dissector of claim 3, further comprising a third pin fixed to the cutting blade and located such that the third pin contacts an outer perimeter of the tool body as the cutting blade moves in the proximal direction from the distal end of the tool body.

5. The tissue dissector of claim 3, wherein the cutting blade is aligned longitudinally along a longitudinal length of the tool body when the second pin is at the farthest distal extent of the path in the tool body.

6. The tissue dissector of claim 3, wherein the cutting blade is aligned at an offset angle from a longitudinal length of the tool body when the second pin is located in the horizontal portion of the path in the tool body.

7. The tissue dissector of claim 3, wherein the handle assembly comprises a finger grip coupled to the central shaft.

8. The tissue dissector of claim 3, wherein the handle assembly includes a bias member disposed in the handle assembly that is compressed as the cutting blade moves in a proximal direction from the farthest distal extent of the path in the tool body.

9. The tissue dissector of claim 3, wherein the cutting blade pivots about the first pin by at least 45 degrees as the second pin travels through the arcuate portion of the path on the cutting body.

10. The tissue dissector of claim 9, wherein the cutting blade pivots about the first pin by up to 90 degrees as the second pin travels through the arcuate portion of the path on the cutting body.

11. The tissue dissector of claim 3, wherein the cutting blade pivots about the first pin by 90 degrees as the second pin travels through the arcuate portion of the path on the cutting body.

12. The tissue dissector of claim 3, wherein the handle assembly includes a palm stop located at a farthest proximal end of the tissue dissector.

13. The tissue dissector of claim 3, wherein the cutting blade is oriented so that a cutting edge can cut tissue as the cutting blade travels in a proximal direction from a farthest distal extent of the tissue dissector.

14. A method of dissecting tissue of a patient, the method comprising:
pivoting a cutting blade about a first pivot pin from a horizontal alignment to an angular offset alignment from the vertical alignment by moving a second pin coupled to the cutting blade through an arcuate portion of a path in a tool body;
moving the cutting blade in the horizontal plane in a proximal direction from the arcuate portion of the path while maintaining the cutting blade in the angular offset alignment by moving the second pin through a horizontal portion of the path in the tool body.

15. The method of claim 14, further comprising, compressing a bias member as the cutting blade moves in the proximal direction from the horizontal alignment.

16. The method of claim 14, further comprising coupling an actuator handle to the blade to selectively move the cutting blade in the proximal direction from the horizontal alignment with a single hand.

17. The method of claim 14, further comprising pulling a finger grip in a proximal direction to move the cutting blade in the proximal direction from the horizontal alignment with a single hand.

18. The method of claim 14, further comprising pushing a palm stop distally towards the cutting blade to selectively move the cutting blade in the proximal direction from the horizontal alignment.

19. The method of claim 14, wherein the step of pivoting the cutting blade about the first pivot pin from the horizontal alignment to the angular offset alignment from the vertical alignment comprises rotating the cutting blade about the first pin by 45 to 90 degrees.

20. The method of claim 14, wherein the step of pivoting the cutting blade about the first pivot pin from the horizontal alignment to the angular offset alignment from the vertical alignment comprises rotating the cutting blade about the first pin by 90 degrees.
